(19) United States
(12) Patent Application Publication
(10) Pub. No.: US 2008/0103092 A1
(43) Pub. Date: May 1, 2008

(54) Methods for Intradermal, Transdermal or Transmucosal Delivery of Biologically Active Substances

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 11/817,919

(22) PCT Filed: Mar. 11, 2005
(86) PCT No.: PCT/RU05/00108

§ 371 Date: Nov. 15, 2007

(51) Int. Cl. A61K 47/02; A61K 38/02; A61K 48/00

(52) U.S. Cl. 514/8; 514/2; 514/769

(57) ABSTRACT

This invention relates to method for intradermal, transdermal or transmucosal delivering a biologically active substance to a mammal in need thereof, which method comprises a step of co-administering to said mammal with the biologically active substance an effective amount of an absorption enhancer, which is water comprising from about 99.760 to about 10 99.999% of light isotopologue ¹H₂ ¹⁶0 and up to 100% of residual isotopologues ¹H₂ ¹⁷0, ¹H₂ ¹⁸0, ¹H₂H¹⁶0, ¹H²H¹⁷0, ¹H₂H¹⁸0, ₂H₂ ¹⁶0, ²H₂ ¹⁷0, and ²H₂ ¹⁸0. Such biologically active substance is selected from the group consisting of drugs, physiologically active peptides, physiologically active proteins, glycoproteins, nucleic acid, nutrients, vitamins, and minerals.

### TECHNICAL FIELD

The present invention relates to human or animal healthcare. More specifically, the present invention relates to method for intradermal, transdermal or transmucosal delivering a bioactive substance to a mammal in need thereof.

### BACKGROUND OF THE INVENTION

Transdermal or transmucosal delivery is an alternative method of systemic delivery of biologically active substances that offers several advantages over both injectable and enteral methods. Biologically active substances that are absorbed through the oral mucosa or skin directly enter the systemic circulation, bypassing the gastrointestinal tract and first-pass metabolism in the liver. This results in rapid onset of action via a more comfortable and convenient delivery route than the intravenous route. However, the transdermal or transmucosal delivery of biologically active substances is limited by barrier function of skin and mucosa that lows absorption of the substance. Especially, the mucosa and skin presents a barrier to the penetration of hydrophilic substances with high molecular weight such as peptides, proteins, and nucleic acids.

Intradermal delivery of biologically active substances into deeper layers of the skin is frequently limited by barrier function of skin. Accordingly, biologically active substances although proved effective for treating skin disorders, have limited effects resulting from only partial penetration into the skin. Especially, the outer layers of skin present a barrier to the penetration of hydrophilic substances with high molecular weight such as peptides, proteins, and nucleic acids.

Thus, there is the great need in effective and safe methods for intradermal, transdermal or transmucosal delivering a biologically active substance to a mammal in need thereof.

Methods for enhancing the intradermal, transdermal and transmucosal delivery of active agents are known in the art. In one approach, promoters of absorption (absorption enhancers) are included in the active agent-containing formulation. Zatz, J. L., Modification of Skin Permeation by Surface-Active Agents in Skin Permeation Fundamentals and Application, 149-162 (1993); and Barry, B. W., “Properties That Influence Percutaneous Absorption in Dermatological Formulations,” Percutaneous Absorption, 127-233 (1983). Surface active agents phosphatidylcholine and sodium glycocholate are examples of absorption promoters. However, addition of these surface active agents results in only a slight to moderate enhancement of permeability at the expense of damaging the barrier tissue.

Thus, there is the great need in effective and safe absorption enhancers for intradermal, transdermal or transmucosal delivering a biologically active substance, especially hydrophilic, to a mammal in need thereof.

Natural water is a composition of nine water isotopologues (¹H₂¹⁶O, ¹H₂¹⁷O, ¹H₂¹⁸O, ¹H²H¹⁶O, ¹H²H¹⁷O, ₁H²H¹⁸O, ²H₂¹⁶O, ²H₂ ¹⁷O, ²H₂ ¹⁸O) formed by stable isotopes of hydrogen (¹H and ²H) and oxygen (¹⁶O, ¹⁷O, ¹⁸O), wherein the level of light water isotopologue ¹H₂¹⁶O is about 99.7317% (Vienna Standard Mean Ocean Water, VSMOW), and wherein total level of all eight heavy isotopologues comprising at least one heavy isotopes ²H, ¹⁷O, and ¹⁸O is about 0.2683% (e.g. 0.199983% ¹H₂¹⁸O, 0.0372% ¹H₂¹⁷O, 0.031069% ¹H²H ¹⁶O, 0.0000623% ¹H²H ¹⁸O, and 0.0000116% ¹H²H ¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, δ, relative to the international VSMOW standard. The natural water enriched maximally by major light water isotopologue ¹H₂¹⁶O was founded in Antarctica (Standard Light Antarctic Precipitation, SLAP), wherein said δ-values of residual heavy isotopes are δ²H −415.5‰, δ¹⁷O-28.1‰, and δ¹⁸O-53.9‰ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50. Thus, water with the abundance of light water isotopologue ¹H₂¹⁶O more than 99.757% is not found in nature.

Complete depletion of natural water of deuterium-comprising isotopologues (¹H²H¹⁶O, ¹H²H¹⁷O, ¹H²H¹⁸O, ²H₂¹⁶O, ²H₂¹⁷O, and ²H₂¹⁸O) provides water enriched by light water isotopologue ¹H₂¹⁶O to the level never more than 99.76%, since total level of these deuterium-comprising isotopologues in water is below 0.031%. Thus, water with content of light water isotopologue ¹H₂¹⁶O more than 99.76% can be prepared in industrial scale by methods providing depletion of natural water of heavy isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

Unexpectedly, we discovered that water enriched by light water isotopologue ¹H₂¹⁶O to the level unknown in nature (from 99.760 to 99.999%) increases absorption of biologically active substances into and through skin and mucosa as compared to water with natural levels of light water isotopologue ¹H₂¹⁶O (from 99.710 to 99.757%).

It is an object of the present invention to provide a method for intradermal, transdermal or transmucosal delivering a biologically active substance to a mammal in need thereof, which method comprises a step of co-administering to said mammal with the biologically active substance an effective amount of an absorption enhancer, which is water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

### BRIEF DESCRIPTION OF THE DRAWINGS

FIG. 1 is a schematic side view of an apparatus for the manufacturing the water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁶O, ¹H²H¹⁸O, ¹H₂¹⁶O, ²H₂¹⁷O, and ²H₂¹⁸O.

### DISCLOSURE OF INVENTION

The present invention provides a method for intradermal, transdermal or transmucosal delivering a biologically active substance to a mammal in need thereof, which method comprises a step of co-administering to said mammal with the biologically active substance an effective amount of an absorption enhancer, which is water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H²H¹⁶O, and ¹H₂¹⁸O.

Herein and after, term “light water” refers to water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H₂H¹⁸O, ²H₂¹⁶O, ²H₂¹⁷O, and ²H₂¹⁸O. Relative amounts of particular heavy isotopologues could vary depending upon the procedure of the preparing the water of the invention, but the sum of residual heavy isotopologues formed by heavy isotopes should not exceed 0.001 to 0.240%. Accordingly, the residual amounts of heavy isotopes in the water of the invention could vary from 0.01 ppm to 155 ppm for ²H, 1 to 360 ppm for ¹⁷O, and 1 to 2000 ppm for ¹⁸O, but the sum of heavy isotopologues formed by these residual heavy isotopes should not exceed 0.001 to 0.240%.

As used herein, the term “intradermal” means that in the method of the invention a therapeutically effective amount of biologically active substance is applied to a mammalian skin for the purpose to deliver this substance to deeper layers of skin and thus achieve a desired therapeutic effect.

As used herein, the term “transdermal” means that in the method of the invention a therapeutically effective amount of biologically active substance is applied to a mammalian skin for the purpose to deliver this substance to systemic circulation and thus achieve a desired therapeutic effect.

As used herein, the term “transmucosal” means that in the method of the invention a therapeutically effective amount of biologically active substance is applied to a mammalian mucosa for the purpose to deliver this substance to systemic circulation and thus achieve a desired therapeutic effect. Nonexclusively, the mucosa is nasal, oral (e.g. buccal or sublingual), gastrointestinal, rectal, or vaginal mucosa.

As used herein, the term “absorption enhancer” means an agent that enhances the penetration of a substance into or through skin or mucosa.

In the method of the invention, the biologically active substance is selected from the group consisting of drugs, physiologically active peptides, physiologically active proteins, glycoproteins, nucleic acid, nutrients, vitamins, and minerals.

Preferably, the drug is selected from the group consisting of analgesic agents, anesthetic agents, antiacne agents, anti-aging agents, antiallergic agents, antiallopecia agents, antianemic agents, antianginal agents, antianxiety agents, antiarrythmic agents, antiasthmatic agents, antibacterial agents, anticancer agents, anticholinergic agents, anticoagulant agents, anticonvulsant agents, antidepressant agents, antidiabetic agents, antidiarrheal agents, antidiuretic agents, antidotes, antidyskinetic agents, antidysmenorrheal agents, antiemetic agents, antifibrinolytic agents, antifibrotic agents, antifungal agents, antiglaucoma agents, antihemorrhagic agents, antihistaminic agents, antihypercalcemic agents, antyhyperglycemic agents, antihyperlipidemic agents, antihypertensive agents, antihyperthyroid agents, antihyperuricemic agents, antihypocalcemic agents, antihypoglycemic agents, antihypotensive agents, anti-inflammatory agents, anti-Kawasaki disease agents, antimalarial agents, antimethemoglobinemic agents, antimigraine agents, antimyastenic agents, antineoplastic agents, antineuralgic agents, antineutropenic agents, antipanic agents, antipolyneuropathy agents, antiprotozoal agents, antipsoriatic agents, antipsychotic agents, antipyretic agents, antirheumatic agents, antiseborrheic agents, antispasmodic agents, antithrombotic agents, antitremor agents, antiulcer agents, antivertiligo agents, antiviral agents, anti-wrinkling agents, appetite stimulants, appetite suppressants, asthma prophylactic agents, bone resorption inhibitors, bronchodilators, contraceptive agents, corticosteroids, depigmentation agents, diuretic agents, eastrogens, hair growth promoting agents, hair growth inhibitor agents, hemapoietic stimulants, hepatitis treatment agents, histamine H2-receptor antagonists, hormones, humectants, immunosuppressants, impotence therapy agents, insulin resistance treatment agent, interferon resistance treatment agent, keratolytic agents, miotic agents, mucolytic agents, mydriatic agents, myocardial infarction therapy or prophylactic agents, neuromuscular blocking agents, nutritional supplements, vitamins, osteoporosis prophylactic or therapy agents, sedative agents, skeletal muscle relaxant, skin emollients and skin moisturizers, skin lightening agents, sympathomimetic agent, thrombolitic agents, vaccines, vasodilators, wound healing promoters, counterirritants, vitamins, nutrients, amino acids and their derivatives, minerals, herbal extracts, retinoids, bioflavonoids, and antioxidants.

Nonexclusive examples of analgesic agent include acetaminophen, hydrocodone bitartrate, codeine, caffeine, acetylsalicylic acid, diclofenac, or mixture thereof.

Nonexclusive examples of anesthetic agent include lidocaine, tetracaine, and epinephrine.

Nonexclusive examples of antiacne agent include erythromycine, tetracycline, benzoyl peroxide, nicotinamide, resorcinol, doxycicline, and salycilic acid.

Nonexclusive examples of anti-aging agents include sunscreens such as octyl-methyl cinnamates and derivatives thereof; retinoids; vitamins such as vitamin C, vitamin B, and derivatives thereof; antioxidants including alpha hydroxy acid such as glycolic acid, citric acid, lactic acid, malic acid, succinic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and mixtures thereof.

Nonexclusive examples of antianemic agent include cyanocobalamine, epoietin, ferrous fumarate, ferrous sulfate, ferrous citrate, and iron dextran.

Nonexclusive examples of antianginal agent include atenolol, isosorbide mononitrate, nitroglycerine, propranolol, timolol, metoprolol, and verapamil.

Nonexclusive examples of antianxiety agent include alprazolam, bromazepam, lorazepam, and diazepam.

Nonexclusive examples of antiarrythmic agent include acebutolol, atenolol, atropine, and metoprolol.

Nonexclusive examples of antiasthmatic agent include dexmethasone, and loratadine.

Nonexclusive examples of antibacterial agent include aminosalycilic acid, isoniazide, erythromycine, gentamicin, ciprofloxacin, amoxicillin, cephaxelin, and streptomycin.

Nonexclusive examples of anticancer agent include daunorubicin.

Nonexclusive examples of anticholinergic agent include atropine.

Nonexclusive examples of anticoagulant agent include heparin, warfarin, and dicumarol.

Nonexclusive examples of anticonvulsant agent include clonazepam, diazepam, and valproic acid.

Nonexclusive examples of antidepressant agent include amitriptyline, and fluoxetin.

Nonexclusive examples of antidiabetic agent include insulin, metformin, succinic acid, and sulfonylureas like as glyburide.

Nonexclusive examples of antidiarrheal agent include codeine, and bismuth subsalycilate.

Nonexclusive examples of antidiuretic agent include vasopressin.

Nonexclusive examples of antidote include glucagon to calcium channel blocking agents, and penicillamine to heavy metals.

Nonexclusive examples of antidyskinetic agent include levodopa.

Nonexclusive examples of antidysmenorrheal agent include diclofenac.

Nonexclusive examples of antiemetic agent include chlorpromazine.

Nonexclusive examples of antifibrinolytic agent include aminocaproic acid.

Nonexclusive examples of antifibrotic agent include potassium aminobenzoate.

Nonexclusive examples of antifungal agent include ketoconazol.

Nonexclusive examples of antiglaucoma agent include carbachol, and pilocarpine.

Nonexclusive examples of antihemorrhagic agent include factor IX, and aminocaproic acid.

Nonexclusive examples of antihistaminic agent include ranitidine hydrochloride, and loratadine.

Nonexclusive examples of antihypercalcemic agent include calcitonin, etidronate and other biphosphonates.

Nonexclusive examples of antyhyperglycemic agent include metformin.

Nonexclusive examples of antihyperlipidemic agent include clofibrate, and fluvastatin.

Nonexclusive examples of antihypertensive agent include atenolol, captopril, lisinopril, and verapamil.

Nonexclusive examples of antihyperthyroid agent include potassium iodide.

Nonexclusive examples of antihyperuricemic agent include allopurinol.

Nonexclusive examples of antihypocalcemic agent include calcium acetate.

Nonexclusive examples of antihypoglycemic agent include glucagon.

Nonexclusive examples of antihypotensive agent include dihydroergotamine.

Nonexclusive examples of anti-inflammatory agent include acetylsalycilic acid, choline salycilate, ibuprofen, diclofenac, indomethacin, and dexamethasone.

Nonexclusive examples of anti-Kawasaki disease agent include immune globulin.

Nonexclusive examples of antimalarial agent include quanidine.

Nonexclusive examples of antimethemoglobinemic agent include methylene blue.

Nonexclusive examples of antimyastenic agent include neostygmine. Nonexclusive examples of antineoplastic agent include asparaginase, carboplatin, daunorubicin, interferon, tamoxifen, and fluorouracil.

Nonexclusive examples of antineuralgic agent include carbamazepine.

Nonexclusive examples of antipanic agent include clonazepam. Nonexclusive examples of antipolyneuropathy agent include immune globulin.

Nonexclusive examples of antiprotozoal agent include chloroquine, and furazolidone.

Nonexclusive examples of antipsoriatic agent include methotrexate.
Nonexclusive examples of antipsychotic agent include carbamazepine.

Nonexclusive examples of antipyretic agent include acetaminophen.

Nonexclusive examples of antirheumatic agent include methotrexate.

Nonexclusive examples of antiseborrheic agent include pyrithione, salycilic acid.

Nonexclusive examples of antispasmodic agent include glucagon.

Nonexclusive examples of antithrombotic agent include acetylsalycilic acid.

Nonexclusive examples of antitremor agent include diazepam.

Nonexclusive examples of antiulcer agent include omeprazole.

Nonexclusive examples of antivertiligo agent include diphenidol.

Nonexclusive examples of antiviral agent include acyclovir, and ribavirin.

Nonexclusive examples of appetite stimulant include dronabinol.

Nonexclusive examples of appetite suppressant include phentermine.

Nonexclusive examples of asthma prophylactic agent include theophylline.

Nonexclusive examples of bone resorption inhibitor include calcitonin.

Nonexclusive examples of bronchodilator include theophylline, and albuterol.

Nonexclusive examples of contraceptive agent include levonorgestrel.

Nonexclusive examples of corticosteroid include dexamethasone.

Nonexclusive examples of skin lightening agents include hydroquinone, catechol and its derivatives, ascorbic acid and its derivatives, and mixtures thereof.

Nonexclusive examples of counterirritants include camphor, menthol, methyl salicylate, peppermint and clove oils, ichtammol, and mixtures thereof.

Nonexclusive examples of diuretic agent include furosemide, and hydrochlorothiazide.

Nonexclusive examples of eastrogen include estradiol.

Nonexclusive examples of hemapoietic stimulant include epoietin.

Nonexclusive examples of hepatitis treatment agent include interferon alpha.

Nonexclusive examples of histamine H2-receptor antagonist include cimetidine.

Nonexclusive examples of hormones include melatonin, thyroid hormones, thyroxine, triiodothyronine, adrenaline, noradrenaline, glucocorticoids (e.g. cortisol), mineralocorticoids (e.g. aldosteron), estrogens (e.g. estradiol), progesterone, androgens (e.g. testosteron), calcitriol, and calciferol.

Nonexclusive examples of immunosuppressant include cyclosporine.

Nonexclusive examples of impotence therapy agent include papaverine.

Nonexclusive examples of keratolytic agent include benzoyl peroxide.

Nonexclusive examples of miotic agent include carbachol.

Nonexclusive examples of mucolytic agent include acetylcysteine.

Nonexclusive examples of mydriatic agent include atropine.

Nonexclusive examples of myocardial infarction therapy or prophylactic agent include acetylsalycilic acid.

Nonexclusive examples of neuromuscular blocking agent include succinylcholine.

Nonexclusive examples of nutritional supplement include calcium lactate, copper gluconate, ferrous fumarate, magnesium chloride, selenous acid, ascorbic acid, beta-carotene, biotin, calcitriol, calcium pantothenate, cyanocobalamine, ergocalciferol, folic acid, niacin, niacinamide, pantothenic acid, pyridoxine, riboflavin, sodium ascorbate, lipoic acid, inositol, and thiamine.

Nonexclusive examples of osteoporosis prophylactic or therapy agent include estrogens.

Nonexclusive examples of sedative agent include diazepam.

Nonexclusive examples of skeletal muscle relaxant include phenyloin.

Nonexclusive examples of thrombolitic agent include urokinase.

Nonexclusive examples of vasodilator include enalapril.

Typically, the drug of the invention is administered in the method of the invention in amounts close to its normal dosage and daily regimen ranges as detailed in medical literature.

Preferably, the physiologically active peptide or protein is selected from the group consisting of cytokines, peptide hormones, growth factors, factors acting on the cardiovascular system, factors acting on the central and peripheral nervous systems, factors acting on humoral electrolytes, hematopoietic factors, factors acting on bone and skeleton, factors acting on the gastrointestinal system, factors acting on the immune system, factors acting on the genital organs, and enzymes.

Nonexclusive examples of cytokines include interferons (e.g. interferon-alpha, -beta, and -gamma) and interleukins (e.g. interleukin 2 through 12).

Nonexclusive examples of protein or peptide hormones include insulin, insulin-like growth factor-1, growth hormone, luteinizing hormone-releasing hormone (LH-RH), adrenocorticotropic hormone (ACTH), amylin, oxytocin, activin, amylin, angiotensin, atrial natriuretic peptides (ANP), calcitonin, cholecystokinin (CCK), ciliary neurotrophic factor (CNTF), corticotropin-releasing hormone (CRH or CRF), erythropoietin, follicle-stimulating hormone (FSH), gastrin, gastrin inhibitory peptide (GIP), gastrin-releasing peptide, ghrelin, glucogon, gonadotropin-releasing factor (GnRF or GNRH), growth hormone releasing hormone (GRF, GRH), human chorionic gonadotropin (hCH), inhibin A, inhibin B, leptin, lipotropin (LPH), luteinizing hormone (LH), motilin, neuropeptide Y, oxytocin, pancreatic polypeptide, parathyroid hormone (PTH), placental lactogen, prolactin (PRL), prolactin-release inhibiting factor (PIF), prolactin-releasing factor (PRF), PYY3-36, secretin, somatostatin (SIF, growth hormone-release inhibiting factor, GIF), thrombopoietin, thyrotropin (thyroid-stimulating hormone, TSH), thyrotropin-releasing factor (TRH or TRF), vasoactive intestinal peptide (VIP), vasopressin (antidiuretic hormone, ADH) and combinations thereof.

Nonexclusive examples of growth factors include nerve growth factors (NGF, NGF-2/NT-3), epidermal growth factor (EGF), fibroblast growth factor (FGF), insulin-like growth factor (IGF), transforming growth factor (TGF), platelet-derived cell growth factor (PDGF), keratinocyte growth factor (TGF), and hepatocyte growth factor (HGF).

Nonexclusive examples of factors acting on the cardiovascular system include which control blood pressure, arteriosclerosis, etc., such as endothelins, endothelin inhibitors, endothelin antagonists, vasoppressin, renin, angiotensin I, angiotensin II, angiotensin III, angiotensin I inhibitor, angiotensin II receptor antagonist, atrial naturiuretic peptide (ANP), and antiarrythmic peptide.

Nonexclusive examples of factors acting on the central and peripheral nervous systems include opioid peptides (e.g. enkepharins, endorphins, kyotorphins), neurotrophic factor (NTF), calcitonin gene-related peptide (CGRP), thyroid hormone releasing hormone (TRH), glial-derived neurotrophic factor (GDNF), brain-derived neurotrophic factor (BDNF), and neurotrophines (e.g. NT3, NT4).

Nonexclusive examples of factors acting on humoral electrolytes include calcitonin.

Nonexclusive examples of hematopoietic factors include erythropoietin, granulocyte colony stimulating factor (G-CSF), granulocyte-macrophage stimulating factor (GM-CSF) and macrophage colony stimulating factor (M-CSF), and thrombopoietin.

Nonexclusive examples of factors acting on the gastrointestinal system include secretin and gastrin.

Nonexclusive examples of factors acting on the immune system include interferons (e.g. interferon-alpha, and -gamma), monoclonal antibody, and naturally occurring, chemically synthesized or recombinant peptides or proteins which may act as antigens. These factors are administered, either independently, as coupled to haptens, or together with an adjuvant, in the formulations according to the present invention.

Nonexclusive examples of factors acting on bone and skeleton include parathyroid hormone and calcitonin.

Nonexclusive examples of factors acting on the genital organs include luteinizing hormone-releasing hormone (LH-RH) and luteinizing hormone.

Nonexclusive examples of enzymes include superoxide dismutase (SOD), urokinase, asparaginase, and kallikreins.

These peptides or proteins may further include glycosylated peptides or proteins, and chemically modified peptides or proteins. Nonexclusive examples of chemically modified peptides or proteins include peptides or proteins linked by a covalent chemical bond to synthetic polymers such as polyethylene glycol, natural polymers such as chondroitin, polysaccharides, etc. and those chemically modified with non-peptide substances. The non-peptide substances mentioned above may be ligands for receptors or antigens for antibodies.

Furthermore, the peptide or protein may be a plurality of peptides joined together by chemical means or by genetic engineering technology.

Nonexclusive examples of nucleic acids include plasmid DNA, RNA and oligonucleotides.

Typically, the peptide, protein or nucleic acid of the invention is administered in the method of the invention in amounts close to its normal dosage and daily regimen ranges as detailed in medical literature for injective dosage forms.

In the method of the invention, the peptide, protein or nucleic acid is administered at the pH range which does not remarkably affect the activity of the physiologically active peptide or protein and is physiologically acceptable. The preferred range is about pH 2 to pH 10. The more preferred range is about 3 to pH 9, and the most desirable range is about pH 3.5 to pH 8. Preferably, buffer of the invention include, but are not limited to, glycine, citrate, succinate, fumarate, malate, or phosphate buffer.

Preferably, the nutrient is selected from the group consisting of carbohydrates, proteins, fats, water, essential fatty acids, dicarboxylic acids, and amino acids (e.g. alanine, arginine, asparagines, aspartic acid, cysteine, glutamine, glutamic acid, glycine, histidine, isoleucine, leucine, lysine, methionine, phenylalanine, praline, serine, threonine, tryptophan, tyrosine, valine and mixtures thereof, and derivatives thereof).

Preferably, the vitamin is selected from the group consisting of thiamin, riboflavin, niacin, pantothenates, pyridoxine, folic acid, cobalamin, biotin, choline, inositol, ascorbic acid, lipoic acid, and carnitine.

Preferably, the mineral is selected from the group consisting of calcium, chromium, cobalt, copper, fluoride, germanium, iodine, iron, lithium, magnesium, manganese, molybdenum, phosphorus, potassium, selenium, silicon, sodium, sulfur, vanadium, and zinc.

In the method of the invention, non-optional ingredients can be co-administered together with the biologically active substance and absorption promoting amounts of light water. Such ingredients include non-exclusively preservatives, flavors, colorants, and absorption promoter known from the art. Such absorption promoter may be any promoter that is pharmaceutically acceptable. Thus, there can be sodium salicylate and salicylic acid derivatives (acetyl salicylate, choline salicylate, salicylamide, etc.), amino acids and salts thereof (e.g. monoaminocarboxlic acids such as glycine, alanine, phenylalanine, proline, hydroxyproline, etc., hydroxyamino acids such as serine etc., acidic amino acids such as aspartic acid, glutamic acid, etc. and basic amino acids such as lysine etc., inclusive of their alkali metal or alkaline earth metal salts), N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts), substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and etc.

In practicing the method of the invention, the biologically active substance and absorption promoting amounts of light water can be co-administered to a mammal in need thereof in a variety of unit dosage form. Such forms include, but are not limited to, gel tablets, aerosols, sprays, syrups, drops, suppositories, lotions, creams, and solutions.

Sublingual dosage form (e.g. sublingual spray, gel tablet, or solution) is applied on sublingual mucosa of the mammal for period at least 10 second and this method can be reapplied from 1 to about 5, preferably from 1 to 3 times per day.

Buccal dosage form (e.g. buccal spray or gel tablet) is applied on buccal mucosa of the mammal for period at least 10 second and this method can be reapplied from 1 to about 5, preferably from 1 to 3 times per day.

Oral dosage form (e.g. syrup, gel tablet) is ingested and the biologically active substance is applied on gastrointestinal mucosa of the mammal and this method can be reapplied from 1 to about 5, preferably from 1 to 3 times per day.

Rectal dosage form (e.g. rectal suppository) is inserted into the anus with fingers and the biologically active substance is applied on rectal mucosa of the mammal and this method can be reapplied from 1 to about 5, preferably from 1 to 3 times per day.

Vaginal dosage form (e.g. vaginal suppository) is inserted into the vagina with fingers or the pertinent applicator and the biologically active substance is applied on vaginal mucosa of the mammal and this method can be reapplied from 1 to about 5, preferably from 1 to 3 times per day.

Nasal dosage form (e.g. nasal spray or drops) is applied on nasal mucosa of the mammal and this method can be reapplied from 1 to about 5, preferably from 1 to 3 times per day.

Topical dosage form (e.g. eye drops) is applied on mucosa surrounded the eye of the mammal and this method can be reapplied from 1 to about 5, preferably from 1 to 3 times per day.

Topical dosage form (e.g. intradermal gel) is applied on skin of the mammal, and is preferably left on the skin for a period of at least about 15 minutes, more preferably at least about 30 minutes, even more preferably at least about 1 hour, most preferably for at least several hours, e.g., up to about 12 hours. This method can be reapplied from 1 to about 5, preferably from 1 to 3 times per day.

Topical dosage form (e.g. transdermal gel) is applied on skin of the mammal, and is preferably left on the skin for a period of at least about 15 minutes, more preferably at least about 30 minutes, even more preferably at least about 1 hour, most preferably for at least several hours, e.g., up to about 12 hours. This method can be reapplied from 1 to about 5, preferably from 1 to 3 times per day.

In practicing the method of the invention, the biologically active substance can be administered to a mammal in need thereof by location the mammalian body to the bath comprising a solution of the substance in light water for a period of at least about 15 minutes.

In practicing the method of the invention, the effective amount of light water is from 0.00001 to 100 kg and depends on the particular method of administering the biologically active substance. In one preferred embodiment, the effective amount of light water is from 0.00001 to 0.01 kg, if the biologically active substance is administered in unit dosage form. In another preferred embodiment, the effective amount of light water is from 1 to 100 kg, if the biologically active substance is administered to a mammal which body is located into the bath filled with a solution of the biologically active substance in light water. Preferably, mammal is human.

In practicing the method of the invention, the biologically active substance and absorption promoting amounts of light water can be co-administered to a mammal in need thereof with using physical processes that provide active diffusion of the substance into or through skin or mucosa. Such physical processes include, but are not limited to, sonophoresis, iontophoresis, microporation, heating, and air pressure.

The following examples are presented to demonstrate the invention. The examples are illustrative only and are not intended to limit the scope of the invention in any way.

### EXAMPLE 1

This example demonstrates the method for producing light water.

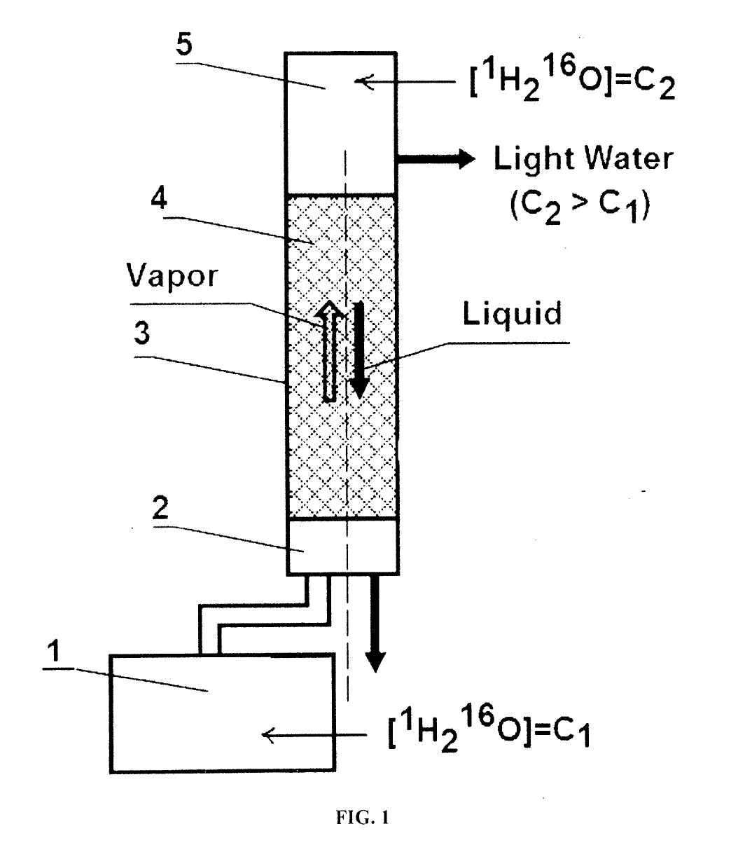

Light water comprising 99.99% of light isotopologue ¹H₂¹⁶O is prepared by highly-effective distillation of natural water comprising 99.73% of light isotopologue ¹H₂¹⁶O with using the apparatus of FIG. 1 under temperature 60° C. and pressure 0.2 bars. The process of the distillation comprises evaporating natural water comprising 99.73% (C₁) of light isotopologue ¹H₂¹⁶O in boiling means 1 to produce water vapor; supplying the water vapor to the bottom 2 of distillation column 3; carrying out vapor-liquid contact between a descending liquid and an ascending vapor mainly on the surface of the contact device 4 (e.g. structured or random packing) within the distillation column, at which time the liquid and the vapor flow in mutually opposite directions over the surface of the contact device along a main flow direction which is along a direction of the column axis; condensing water vapor with concentration of light isotopologue ¹H₂¹⁶O 99.99% (C₂) on condenser 5 installed on upper bound of the distillation column 3; and collecting a part of condensate as condensed raw light water comprising 99.99% of light isotopologue ¹H₂¹⁶O (C₂>C₁) appropriate for producing light pharmaceutical water. Then, the raw light water is treated consequently by procedures of filtration, disinfection, deionization, and ultra-filtration. Resulted water comprising 99.99% of light isotopologue ¹H₂¹⁶O can be used in the method of the invention.

### EXAMPLE 2

This example demonstrates the method of transdermal delivery.

Material. Antimethemoglobinemic agent, methylene blue (MB) was used.

Treatment. A solution of methylene blue (100 μg/mouse in 100 μl) prepared on water (99.73% of light isotopologue ¹H₂¹⁶O, control) or light water (99.99% of light isotopologue ¹H₂¹⁶O) was administered topically on skin depilated area (about 1 cm²) of C57BL mice. Methylene blue levels in plasma were measured at 10 min after the administration. Data are presented in Table 1 as methylene blue plasma level mean ±SD (n=5) in percent of control.

Table 1 demonstrates that light water much mores effective as the absorption promoter for transdermal delivery as compared to water.

### EXAMPLE 3

This example demonstrates the method of transmucosal delivery.

Material. Human recombinant interferon-alpha (IFN) was used.

Treatment. Male C57B1 mice about 20 g weights were treated sublingually with 170 ng/mouse IFN in form of 20 μl/mouse solution made on water (99.73% of light isotopologue ¹H₂¹⁶O, control) or light water (99.99% of light isotopologue ¹H₂¹⁶O) in citrate buffer under pH 5.4-5.5. Plasma IFN levels were measured at 7 min with immunoenzyme assay specific to human IFN. Data are presented in table 2 as plasma IFN mean ±SD (n=6) in ng/ml.

### EXAMPLE 4

This example demonstrates the method of transdermal delivery.

Material. Human recombinant interferon-alpha (IFN) was used.

Treatment. Male C57B1 mice about 20 g weights were depilated 72 hours before the experiment. 100 ng/mouse IFN in form of 100 μl/mouse solution made on water (99.73% of light isotopologue ¹H₂¹⁶O, control) or light water (99.99% of light isotopologue ¹H₂¹⁶O) in succinate buffer under pH 5.1 were applied on skin depilated area of about 1 cm². Plasma IFN levels were measured at 10 min with immunoenzyme assay specific to human IFN. Data are presented in table 3 as plasma IFN mean ±SD (n=5) in ng/ml.

### EXAMPLE 5

This example demonstrates the method of transmucosal delivery.

Material. Ovalbumin as antigen protein was used.

Treatment. Male C57B1 mice about 20 g weights were treated sublingually with 100 μg/mouse ovalbumin in form of 20 μl/mouse solution made on water (99.73% of light isotopologue ¹H₂¹⁶O, control) or light water (99.99% of light isotopologue ¹H₂¹⁶O) for the days 1 through 3 (the first immunization) and singly at day 15 (the second immunization). Total level of IgG measured in plasma of mice at day 21 was used an index of antigen delivery into circulation under immunization. Data are presented in table 4 as reciprocal Log₅ titers of mouse IgG plasma IFN mean ±SD (n=5) in ng/ml.

### EXAMPLE 6

This example demonstrates the method of transmucosal delivery.

Material. Human recombinant insulin was used.

Treatment. Male adult rats with alloxan diabetes were assigned into two groups: a control rats (n=5) and experimental rats (n=5). Average fasting blood glucose levels in rats were 10.0±0.2 mmol/l. Rats were treated sublingually with 0.1 mg/mouse insulin dissolved on water (99.73% of light isotopologue ¹H₂¹⁶O, control) or light water (99.99% of light isotopologue ¹H₂¹⁶O) for 10 min. Blood glucose levels were measured as an index of insulin delivery into circulation. Data are presented in table 5 as blood glucose level mean ±SD (n=5).

### EXAMPLE 7

This example demonstrates the method of transdermal delivery.

Material. Human recombinant insulin was used.

Treatment. Male adult rats with alloxan diabetes were shaved 48 hours before the experiment. Rats assigned into two groups: a control rats (n=5) and experimental rats (n=5). Average fasting blood glucose levels in rats were 11.0±0.4 mmol/l. 0.1 mg/mouse insulin dissolved on water (99.73% of light isotopologue ¹H₂¹⁶O, control) or light water (99.99% of light isotopologue ¹H₂¹⁶O) was applied on depilated skin for 10 min. Blood glucose levels were measured as an index of transdermal insulin delivery into circulation. Data are presented in table 6 as blood glucose level mean ±SD (n=5).

### EXAMPLE 8

This example demonstrates the method of delivery of the agent to skin cells.

Material. Methyl red dye was used.

Cell culture. Human keratinocytes A431 were used.

Treatment. A431 keratinocytes were incubated for 20 min with 0.3 mg/ml methyl red dissolved in water (99.73% of light isotopologue ¹H₂¹⁶O, control) or light water (99.99% of light isotopologue ¹H₂¹⁶O). Methyl red uptake by cells was measured. Data are presented in Table 7 as methyl red uptake means ±SD (n=8) in percent to control.

### EXAMPLE 9

This example demonstrates the method of intradermal delivery.

Material. Methylene blue (MB) was used.

Treatment. A solution of methylene blue (100 μg/mouse in 100 μl) prepared on water (99.73% of light isotopologue ¹H₂¹⁶O, control) or light water (99.99% of light isotopologue ¹H₂¹⁶O) was administered topically on skin depilated area (about 1 cm²) of C57BL mice. Skins were washed out and methylene blue levels in skin were measured at 5 min after the administration. Data are presented in Table 8 as methylene blue skin level mean ±SD (n=5) in percent of control.

Table 8 demonstrates that light water much mores effective as the absorption promoter for intradermal delivery as compared to water.

TABLE 1
Effect of light water on transdermal delivery of methylene blue.

| | Plasma methylene |
|---|---|
| Treatment | blue, in % |
| Water (99.73% of light isotopologue H₂¹⁶O) | 100 ± 112 |
| Light water (99.99% of light isotopologue | 816 ± 73* |
| ¹H₂¹⁶O) | |

*Differs significantly of control (p < 0.01)

TABLE 2
Effect of light water on sublingual delivery of IFN.

| Treatment | Plasma IFN, ng/ml |
|---|---|
| Water (99.73% of light isotopologue H₂¹⁶O) | 3.7 ± 2.0 |
| Light water (99.99% of light isotopologue | 15.0 ± 1.8* |
| ¹H₂¹⁶O) | |

*Differs significantly of control (p < 0.01)

TABLE 3
Effect of light water on transdermal delivery of IFN.

| Treatment | Plasma IFN, ng/ml |
|---|---|
| Water (99.73% of light isotopologue H₂¹⁶O) | 0.37 ± 0.09 |
| Light water (99.99% of light isotopologue | 3.62 ± 1.01* |
| ¹H₂¹⁶O) | |

*Differs significantly of control (p < 0.01)

TABLE 4
Effect of light water on sublingual delivery of antigen protein.

| Treatment | Reciprocal Log₅ Titers IgG |
|---|---|
| Water (99.73% of light isotopologue H₂¹⁶O) | 0.8 ± 0.4 |
| Light water (99.99% of light isotopologue | 3.8 ± 0.4* |
| ¹H₂¹⁶O) | |

*Differs significantly of control (p < 0.01)

TABLE 5
Effect of light water on sublingual delivery of insulin.

| Treatment | Blood glucose, mmol/l |
|---|---|
| Water (99.73% of light isotopologue H₂¹⁶O) | 9.7 ± 0.5 |
| Light water (99.99% of light isotopologue | 7.3 ± 0.3* |
| ¹H₂¹⁶O) | |

*Differs significantly of control (p < 0.05)

TABLE 6
Effect of light water on transdermal delivery of insulin.

| Treatment | Blood glucose , mmol/l |
|---|---|
| Water (99.73% of light isotopologue H₂¹⁶O) | 10.7 ± 0.6 |
| Light water (99.99% of light isotopologue | 8.3 ± 0.5* |
| ¹H₂¹⁶O) | |

*Differs significantly of control (p < 0.05).

TABLE 7
Effect of light water on methyl red
delivery into A431 keratinocytes.

| | Methyl red uptake, in % of |
|---|---|
| Treatment | control |
| Water (99.73% of light isotopologue H₂¹⁶O) | 100 ± 4 |
| Light water (99.99% of light isotopologue | 132 ± 4* |
| ¹H₂¹⁶O) | |

*Differs significantly of control (p < 0.01)

TABLE 8
Effect of light water on intradermal delivery of methylene blue.

| | Plasma methylene |
|---|---|
| Treatment | blue, in % |
| Water (99.73% of light isotopologue H₂¹⁶O) | 100 ± 108 |
| Light water (99.99% of light isotopologue | 1715 ± 285* |
| ¹H₂¹⁶O) | |

*Differs significantly of control (p < 0.01)

We claim:

1. A method for intradermal, transdermal or transmucosal delivering a biologically active substance to a mammal in need thereof, which method comprises a step of co-administering to said mammal with the biologically active substance an effective amount of an absorption enhancer, which is water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

2. The method according to claim 1, wherein the biologically active substance is selected from the group consisting of drugs, physiologically active peptides, physiologically active proteins, glycoproteins, nucleic acid, nutrients, vitamins, and minerals.
